(19) United States
(12) Patent Application Publication
(10) Pub. No.: US 2004/0096943 A1
(43) Pub. Date: May 20, 2004

(54) Method and device for cultivating of cells at high densities and for obtaining of products from these cells

(76) Inventors: Uwe Marx, Berlin (DE); Marco Riedel, Berlin (DE); Hikmat Bushnaq-Josting, Berlin (DE)

Correspondence Address: Horst Kasper, 13 Forest Drive, Warren, NJ, 07059, US

(21) Appl. No.: 10/622,502

(22) Filed: Jul. 18, 2003

Related U.S. Application Data
(60) Provisional application No. 60/397,083, filed on Jul. 19, 2002.

(30) Foreign Application Priority Data
Jan. 28, 2002 (DE) 102 04 382.5

(51) Int. Cl. C12P 21/02; C12N 5/06; C12N 5/04

(52) U.S. Cl. 435/69.1; 435/325; 435/419

(57) ABSTRACT

The invention relates to a method and a device which allow to cultivate cells in a sterile way over long time periods and at high densities and to obtain the maximum product volumes for time units from the cell cultures. Here the cultivation chamber and the supply container are semipermeably separated from each other, wherein the cultivation chamber comprises several fixed chambers and wherein the supply chamber is connected to a device for generating of a variably adjustable gas/cell cultivation medium mixture.

### RELATED APPLICATIONS

This application is a complete application related to Provisional application having application No. 60/397,083 and filed on Jul. 19, 2002.

### BACKGROUND OF THE INVENTION

1. Field of the Invention

The invention relates to method and device, which method and device allow to cultivate cells under sterile conditions over long time periods and at high density and to obtain the maximum product volume per time unit from the cell cultures.

2. Brief Description of the Background of the Invention Including Prior Art

Many different cell cultivation devices for the most different fields of tasks have been developed up to the present point in time. An economically relevant field is the cultivation of cells for the production of medicines and pharmaceutical preparations. At the present time cells are predominantly kept in cultivation according to two methods different in principle:

1. The suspension cultivation in commercial sterile agitator vessel bio reactors.

2. The stationary cultivation at high cell densities, which became possible mostly through the presence of suitable separation membranes and which was for the first time described by Knazek et al. in (Knazek R. A. “Solid tissue masses formed in vitro formed cells cultured on artificial capillaries.” Federation Proc. 33 (8): pp. 1978-1981 (1974) as well as in U.S. Pat. No. 3,821,087. Also flat membranes as described by Scheirer and Katinger (1985, German printed Patent document 3,409, 501) were here employed in addition to cultivation systems out of hollow fiber membranes. The uniform nutrient supply and in particular the oxygen supply are a problem in the recited methods and devices. Both the attempt to solve this problem through complex method steps with the pressure application (1989, U.S. Pat. No. 4,804,628), as well as the direct entry of oxygen into the cell cultivation chamber through a further membrane system (1986, German printed Patent document 2,431, 450 and 1995, German printed Patent document 4,230,194) did not lead to cultivation systems enlargeable at an arbitrary scale and where the cells can be supplied uniformly. Membrane methods are associated with several advantages in comparison to conventional suspension cultivation. The membrane methods can reach very high cell densities 10⁷-10⁸ cells per ml through their operation as perfusion cultures by a large membrane face per volume unit. In addition the cells are protected against damaging shearing forces, require less nutrient materials and exhibit a higher product concentration (Piret J. M. and Cooney C. L.: “Immobilized mammalian cell cultivation in hollow fiber bio reactors.” Biotechnol. Adv. 8: pp. 763-783; 1990). In addition, both suspension cells as well as adherent cells can be cultivated with hollow fibers (Lipman, N. S. and Jackson, L. R.: “hollow fiber bio reactors: an alternative to murine ascites for small-scale (<1 gram) monoclonal antibody production.” Res Immunol July-August; 149(6): pp. 571-576; 1998).

The enlargement of scale is a problem of the high cell density cultivation: more pronounced as compared to the agitator vessel fermentation, the supply of the cells with the nutrients such as oxygen coming from the gas phase and the discharge of metabolites (before the concentration locally reaches toxic values) is a principal condition for the success of the cell cultivation at high cell density systems.

The enlargement of scale is limited by the length of the hollow fibers in connection with hollow fiber bundle bioreactors, wherein the cells are cultivated between the hollow fibers and wherein the nutrients are transported in the lumen of the fiber. The length of the hollow fibers is however limited by the usage of the oxygen from the hollow fibers. An enlargement of scale is thereby only possible by parallelizing. In practice, this leads to uneconomic processes, that is the scalability of the hollow fiber bio reactors fails based on an adequate homogeneous supply of the cells with fresh gas and liquid nutrient components.

Several commercially available systems are based on the hollow fiber technology such as for example CellPharm®, Cellmasx®, or Technomouse®. As an alternative thereto, systems with flat membranes such as CELLine®, miniPERM®, or OptiCell® are commercially offered. The limitations are part of all systems.

The employment of fog as a nutrient source as described for the first time in the Patent of Weathers and Giles of 1989 (U.S. Pat. No. 4,857,464) is another possibility to bring oxygen at high concentrations into the cell cultivation chamber. This method is commercially employed up to present exclusively for the cultivation of Hairy Roots. Hairy Roots are root like plant cells transformed with Agrobacterium rhizogenes and plant secondary metabolites are produced with Hairy Roots (Flores H. E., Hoy M. W. and Pickard, J. J.: “Secondary metabolites from root cultures”, TibTech. 5: p 64-69; 1987). The invention proposed by Weathers and Giles is associated with the disadvantage that mammalian cells cannot be cultivated at high density. This is based on the one hand on the fact that a rolled up grid is proposed for the fixation of the animal cells at the support, which grid is to support the cells. This grid would not be suitable to retain cells at high density which are cultivated in an individual cell culture. At least further steps are required in order to improve the adhesion of the cells, such as for example adhesives or a covalent bond to the carrier. A flow out of cells is to be prevented according to this Patent by the employment of a pillow like device. More detailed information is not presented relative to this pillow. The supply of the cells with oxygen represents a further problem of this invention. Intact cell associations are characterized in that they have active mechanisms for the transfer of the nutrients inclusive oxygen from the outer edge of the cell association toward the interior. The problem of the oxygen supply is not present in the recited Patent, since the cultivation at high density is not planned. Nevertheless, the supply of the cells with oxygen appears to be a problem upon the employment of the pillow like device, at least for the cells in the interior of the pillow like device. A further disadvantage is the obtaining of product according to U.S. Pat. No. 4,857,464 to Weathers and Giles. The products are collected together with the nutrient liquid in the region of the lower chamber and then have to be separated in a product collection container. Also the feeding and removal of cells is relatively complicated.

The reactor of Weathers and Giles does not allow a cultivation of mammalian cells at high densities and does not permit a good separation of a product. A reason for this situation is that a rolled up grid is proposed by Weathers and Giles for fixation of the cells, wherein the rolled up grid is to carry the cells. This rolled up grid would not be suitable to retain cells of a high density, which are grown in individual cell culture. At least additional steps might be required in order to improve the adhesion of the cells, such as for example by way of adhesives or by way of a covalent binding or connection to the carrier. Weathers and Giles intend to prevent a flow out of cells by employing a pillow like device. However, Weathers and Giles do not give detailed information relating to the pillow like device.

The feeding of oxygen to the cells represents a further problem of the teaching of Weathers and Giles. Intact and operating cell compounds are characterized in that they show active steps and mechanisms for transporting of nutrients including oxygen from the outer edge of the cell compound to the interior of the cell compound. Thus the cell compounds of Weathers and Giles are deficient.

This explains why the described mist reactors—designated in the English language as nutrient mist reactors (NMB)—are successfully employed up to today nearly exclusively for the cultivation of Hairy Roots, with the exception of—the only animal cells—amebocites (Liu, C. Z., Wang, Y. C., Zhao, B., Guo, C., Ouyang, F., Ye, H. C., Li, G. F. “Development of a nutrient mist bio reactor for growth of hairy roots.”, In Vitro Cell Dev Biol-Plant May-June; pp. 271-274; 1999), (Wyslouzil, B. E., Whipple, M., Chatterjee, C., Walcerz, D. B., Weathers, P. J., Hart, D. P.:“Mist deposition onto hairy root cultures: aerosol modeling and experiments.”, Biotechnol. Prog. March-April; 13 (2): pp. 185 through 194; 1997) and (Friberg, J. A., Weathers, P. G., Gibson D. G.: “Culture of amebocytes in a nutrient mist bio reactor.”, In Vitro Cell Dev Biol. March; 28A(3pt1): pp. 215-217; 1992).

These amebocytes are produced in the gill lamella of the limulus crustacean and are not cultivated as individual cell cultures in contrast to the animal cell cultures in a conventional sense.

Liu et al. describe the development of a nutrient mist bio reactor for the production of artemisinin (a potential anti-malaria agent) in various embodiments based on an “Inner loop”, that is based on internally circulation, which is comparable with an airlift reactor. For this purpose a 2,3 liter reactor was equipped with three floors of stainless-steel grids (2 mm pore size) as a growth surface for the roots and different strategies were selected for spraying the nutrient.

The mist was generated with a “Transducer—ultra sound” and was periodically sprayed.

Ideal conditions are reached with the variation (c.), since last air was required for the distribution of the aerosol. The productivity of the thus cultivated hairy Roots (variation c) after 25 days was comparable to the productivity of submersed cultures after thirty days.

Wyslouzil et al. investigated the influences of the aerosol transport and of the aerosol deposition at the same kind of hairy Roots. Here a mathematical model was produced for the deposition at the cells, the physical mechanisms of the particle capturing by the root cells (diffusion) were investigated and finally the model was compared with the experimental results. It was determined that the mist having particle diameters of between 3 and 15 micrometers and generated by the ultrasound can penetrate perfectly deep in a dense root bed.

### SUMMARY OF THE INVENTION

### 1. Purposes of the Invention

It is an object of the invention to eliminate the disadvantages of the described bio-reactors and to allow new paths of resolving the cultivation of mammal cells of high cell density.

### 2. Brief Description of the Invention

The object is accomplished by a method for the cultivation of cells in high density and for the obtaining of products from these cells, which method comprises the following steps:

entering of the cells of high density into a cultivation chamber, wherein the cultivation chamber comprises several fixed chambers and wherein the cultivation chamber is semipermeably separated from the supply chamber

supplying of the cells through the supply chamber with a variably adjustable gas/cell cultivation medium mixture

obtaining of the products by the discharge of the products at the semipermeable walls of the chambers and separation of the products based on the gravity of the products.

It was surprisingly found that the method according to the present invention allows to eliminate all disadvantages of the conventional cell culturing chambers and that cultivation systems arbitrarily enlargeable in scale can be provided. The semipermeable separation of cultivation chamber and supply chamber effects that also cells of high density cannot exit from the chambers. The supply of the cells with a variably adjustable gas/cell cultivation medium mixture is associated with the advantage that nutrients diffuse into the cell culture at the semipermeable separation layer. At the same time the cells in the interior of the chamber are supplied sufficiently with oxygen. The cell products exit at the semipermeable separation layer from the chamber, form droplets and fall downwardly based on their gravity.

According to a preferred embodiment each chamber is formed such that the chamber does not extend over a length of 5 mm in one dimension. Otherwise the forms of the chambers are freely scalable. The cells are entered by way of carriers through a central charging system disposed outside of the supply chamber. The application of cells within the supply chamber can therefore be dispensed with. Consequently, additional fixation agents such as adhesives or a covalent bond to the carrier material are therefore not necessary. Preferably membranes are employed as semipermeable separation layer between cultivation chamber and supply chamber. Flat membranes or hollow fiber membranes have proven to be advantageous. The membranes comprise polymers, for example polycarbonate.

Mist, fog, or a spraying or mixture of fine gas bubbles in liquid are employed as a gas/cell cultivation media mixture. The mist can be generated for example by way of ultrasound.

The droplets sinking downward based on their gravity are collected in a vessel for gaining the cell products, wherein the vessel is advantageously disposed directly below the chambers. A product reservoir is connected to this vessel, wherein the product reservoir is disposed outside of the supply chamber. This arrangement according to the present invention is associated with the advantage relative to the known state of the arts in that the withdrawal of the product is performed outside of the supply chamber. In addition it is accomplished that the medium containing product is collected in the vessel disposed below the chambers and separation between medium containing product and medium not containing product is accomplished thereby according to the present invention. The medium not containing product is collected at the edges of the system and at the floor and is returned again to the gas/cell cultivation medium mixture generator.

Plant cells and animal cells (mammalian cells) can be cultivated with the method according to the present invention.

The arrangement according to the present invention comprises a cultivation chamber with several fixed chambers and supply chamber with a device for the generating of a variably adjustable gas/cell cultivation medium mixture, wherein the cultivation chamber is semipermeably separated from the supply chamber.

A collection vessel disposed below the chambers and connected to a product reservoir serves for a separation of the products from the nutrient liquid.

Preferably a mist chamber serves as a device for the generation offered gas/cell cultivation medium mixture. The mist can be generated by ultrasound. Flow meter, temperature measurement apparatus, air filter, pressure gauge, pressure measurement apparatus, fog deposit filters, and condensate catch containers belong furthermore to the invention device.

The method according to the present invention and the apparatus according to the present invention are extremely suitable for the obtaining of proteins, of effective agents and of medicines.

The invention and the functioning of the invention are to be illustrated by way of figures in the following. The figures serve only for the better understanding and the figures, however, are not to be considered as the single possible construction way by which the claims of the present application can be realized.

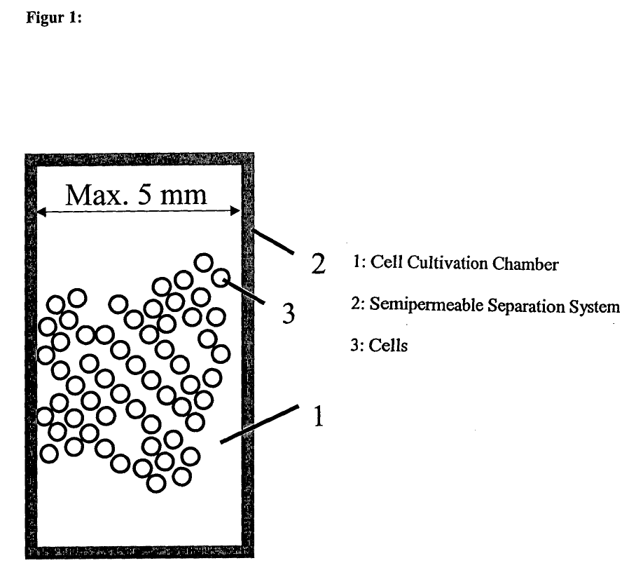

FIG. 1: is a view of a schematic diagram of a cell cultivation chamber,

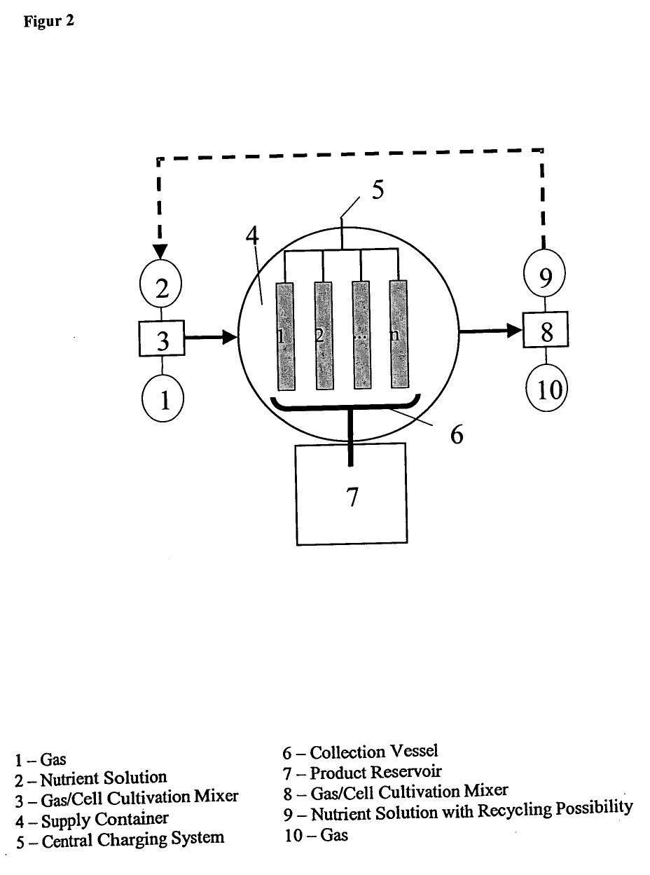

FIG. 2: is a view of a schematic diagram of a central feeding system for all cell cultivation chambers,

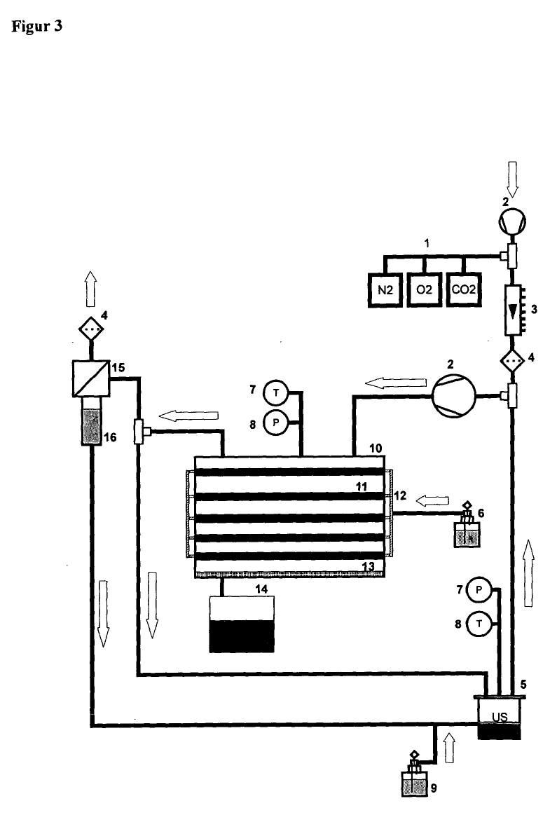

FIG. 3: is a view of a schematic diagram of the flows associated with a central feeding system for all cell cultivation chambers.

### DESCRIPTION OF INVENTION AND PREFERRED EMBODIMENTS

FIG. 1 shows one of many identical cell cultivation chambers in a supply chamber or supply container, wherein the cell cultivation chamber (1) of each chamber is separated from the surrounding supply container by a semipermeable separation system (2) and wherein the chambers are less than or equal to a size of 5 mm in one dimension.

FIG. 2 shows a central feeding system for all cell cultivation chambers (5), catch device for the separated product (6) with a connected product reservoir (7), a mixing station (3) for the production of the gas/cell cultivation medium mixture with a connection to the supply container and with a separating station (8) connected to the supply container for the separation of the gas/cell cultivation medium mixture into its components.

U.S. Pat. No. 4,857,464 teaches a bioreactor. Cells disposed above a supply chamber in a bioreactor with a variably adjustable mixture of a gas and cell culture media. Mist reactors however have not been employed for a cultivation of mammalian cells. The present invention enables for a first time an individual cell cultivation of mammalian cells. An important aspect of the invention is that the cultivation chamber is semipermeably separated from the supply chamber. Semipermeably means that materials and nutrients can pass through a semipermeable wall, where complete cells are unable to pass the semipermeable wall. This produces an exchange of nutrients and of formed products through the semipermeable wall or membrane. The cell culture have to be placed in several small chambers. In case of large chambers there exists the danger, that the cells in the inside of the chamber are supplied insufficiently. For this reason the chambers are not permitted to surpass a length of more than 5 millimeters in one dimension.

It is a feature of the present invention that cells of high density are cultivated at all in a mist reactor. It is a further feature of the invention that the mist reactor employs membranes and a plurality of fixed chambers. Yet another feature of the present invention is the separation and obtaining and exiting of the product by having the product pass through a membrane.

The cells are uniformly distributed onto all chambers in a sterile way based on the central feeding system.

At the same time the supply chamber is continuously flown through with a corresponding gas/cell cultivation medium mixture. The arrangement allows the position of nutrient solution at the surfaces of the chambers. The excessive gas/cell cultivation medium mixture flows out of a discharge outlet into the separating station, wherein the cell cultivation medium is separated from the gas in the separating station and wherein the cell cultivation medium can again be fed to the mixing station if required.

A continuous exchange of nutrients and formed products occurs between the nutrient solution at the surface of the chamber and the cell cultivation chamber such that the cells are supplied continuously with fresh nutrients and the metabolic products and the product exit from the chamber.

The nutrient solution enriched with products and the metabolic products drops into a collection vessel (6). The enriched nutrient solution is collected from the collection vessel (6) into a product reservoir (7). A simple separation of the product harvest from me gas/cell cultivation medium mixture is thereby assured.

This device allows a scale enlargement by multiplying the number of chambers while maintaining a common supply chamber.

No active but only a diffuse transport of nutrients occurs between the cells in the individual cell cultivation according to the present invention.

The features of the invention can be gathered from the elements of the claims and of the description, wherein both individual features as well as several features in the shape of combinations represent advantageous embodiments for which protection is applied with this document.

The essence of the invention comprises a combination of known elements (reactors with a gas/cell cultivation medium) and new elements (separation of cultivation chamber and supply chamber, cultivation chamber composed out of a plurality of chambers, simple product obtaining), which are mutually influenced by each other and result in their new overall effect in an advantage of usage and into the desired success, wherein the success comprises that for the first time a possibility is furnished for cultivation of cells at high density and for obtaining of product from the cells at a cultivation system arbitrarily enlargeable in scale.

The invention is to be illustrated by way of embodiment examples without being limited to these examples.

### EMBODIMENT EXAMPLES

### Example 1

Cassettes were employed as chambers, wherein the cassettes are limited by two sides with porous membranes disposed parallel to each other wherein the distance between the membranes was less than 5 mm. The gas/cell cultivation medium mixture comprised out of fogged cell cultivation medium in a carbon dioxide/room air atmosphere. Six chambers with a cell concentration of in each case 21 million hybridoma cells per milliliter were employed. In each case two chambers were removed on the fourth day and four chambers were removed on the eighth day of the sterile cultivation maintaining and running from the supply chamber and were investigated with respect to the number of cells.

The results are presented in the following table as an average value with an indication of the deviation (n=2 on day four and n=4 on day 8).

Cultivation time (days)

| | 4 | 8 |
|---|---|---|
| cell cultivation (10⁶ cells | 1.68 ± 0.4 | 36.6 ± 0.26 |
| per milliliter) | | |

After an expected fall of the cell concentration, caused by adaptation difficulties to a new environment, to a value of 1.68 million cells per milliliter, the cells could recover in a bio reactor and could reach and surpass again the starting concentration within the following four days. The final concentration of 36.6 million cells per milliliter does not only mean that the high cell concentration could be obtained, but that an increase of the cell concentration was possible by a factor of 1.74. If one considers the results of the days four and eight, then the number of cells has increased by a factor of 22 within the time period of 4 days.

### Example 2

For this purpose the two chambers as described in example 1 were employed. The gas/cell cultivation medium mixture comprised a solution fogged into room air.

The two chambers are in each case supplied with 1.2 mg antibodies in solution. The solution dripping from the chambers was fractioned, in a common drainer vessel collected and was investigated with respect to product by way of ELISA technology. The collection in a common drainer vessel corresponds to the average value formation. The following table presents the dependence of the observed product accumulation in the drainer vessel with respect to time.

| | | antibody | |
|---|---|---|---|
| | | concentration | total mass |
| | fractions | (micrograms per | antibody |
| Time (minutes) | (milliliter) | milliliter) | (micrograms) |
| 0 | 0 | 0 | 0 |
| 5 | 1 | 2.8 | 3 |
| 10 | 2 | 2.8 | 8 |
| 15 | 2 | 2.8 | 14 |
| 20 | 2 | 3.5 | 21 |
| 25 | 2.3 | 3.5 | 29 |
| 30 | 2 | 3.5 | 36 |
| 35 | 2.3 | 4.8 | 47 |
| 40 | 2 | 4.8 | 57 |
| 45 | 2 | 4.8 | 66 |
| 50 | 2.3 | 6.7 | 82 |
| 55 | 2 | 6.7 | 95 |
| 60 | 2.3 | 6.7 | 111 |
| 75 | 6 | 7.7 | 157 |
| 94 | 8 | 8.3 | 223 |

It was possible to obtain already 10 percent of the charged product separated from the supply stream within 1.5 hours.

The example confirms the effective separation of product harvest and supply stream within the invention apparatus.

### Definitions and Abbreviations

Individual cells: individual cells are cells, which do not appear in the intact tissue association

Uniform supply: a supply is uniform according to the present invention, wherein the supply is identical in two dimensions and sufficient in the third dimension and is identical in the percentage composition.

Products: products are cell components according to the present invention, viruses as well as effective agents produced in or through cells.

Semipermeable separation system: the boundary layer between supply chamber and cell cultivation chamber is designated as a semipermeable separation system according to the present invention, wherein the boundary layer is characterized by retaining of cells while exhibiting permeability for products and nutrients.

Stationary cultivation: a not active agitated cultivation is a stationary cultivation according to the present invention.

Carrier system: a carrier system is according to the present invention a medium, which is employed for the entry and/or the support of cells in the cell cultivation chamber. This medium can be liquid, semisolid or also solid in its properties.

Cells: natural cells and accidental or by way of manipulation degenerated cells of any kind of species are designated with the term cell.

Cells at high density are according to the present invention concentrations of individual cells in a stream and cultivation of more than 10 million cells per ml.

1. Method for cultivation of cells at high densities and for obtaining of products from these cells characterized by the following steps:
entering of the cells at high density into a cultivation chamber, wherein the cultivation chamber comprises several chambers and is semipermeably separated from the supply container;
feeding of the cells through the supply container with a variably adjustable gas/cell cultivation medium mixture;
obtaining the products and their discharge at the semipermeable walls of the chambers and based on a dissolution of the products in the variably adjustable gas/cell cultivation medium mixture.

2. Method according to claim 1 characterized in that plant cells and mammalian cells furnish the cells at high density.

3. Method according to claim, characterized in that the bringing in of the cells is performed through a central feeding system disposed outside of the supply container and wherein the cultivation chamber and the supply chamber are such connected to each other that the simultaneous entry of cells in all chambers is possible through a single connection.

4. Method according to one of claims 1 through 3 characterized in that membranes serve for the separation of cultivation chamber and supply container.

5. Method according to claim 4 characterized in that flat membranes or hollow fiber membranes are employed as membranes.

6. Method according to claim 4 characterized in that the membranes comprise a member of the group consisting of poly sulfone, polyethersulfone, polycarbonate, and mixtures thereof.

7. Method according to claim 1 characterized in that a member of the group consisting of a mist having a droplet size of from about 0 to 100 micrometers, a spraying having droplet sizes from about 100 to 5000 micrometers, a mixture out of fine gas bubbles in liquid, and mixtures thereof is employed as a gas/cell cultivation medium mixture.

8. Method according to claim 7 characterized in that the mist is generated by ultrasound with a high frequency; and that the spraying is generated by way of nozzles or spray heads.

9. Method according to one of the claims 1 through 8 characterized in that the products exiting at the membrane are collected in a vessel disposed below the chambers for the obtaining of cell products.

10. Method according to claim 1 characterized in that the gas/cell culture media mixture enriched with product drops off the chambers by gravity and wherein the gas/cell culture media mixture enriched with product is collected below the chambers.

11. Apparatus for cultivation of cells at high densities and for obtaining of products from these cells, comprising a cultivation chamber and a device for generating a variably adjustable gas/cell culture media mixture,
wherein the cultivation chamber is furnished with several fixed chambers and wherein the several fixed chambers are semipermeably separated from a supply container, wherein the supply container contains a variably adjustable gas/cell cultivation medium mixture, wherein vessels for collecting the cell products are disposed below the chambers.

12. Apparatus according to claim 11 characterized in that each of the chambers does not surpass the length of 5 mm in one dimension.

13. Apparatus according to claim 11 characterized in that the cultivation chamber and the supply container are connected such to each other that the simultaneous entering of the cells into all chambers is possible through a single connector.

14. Apparatus according to one of the claims 13 through 14 characterized in that a hose or a Luer-connection serves as the connector.

15. Apparatus according to one of the claims 12 through 14 characterized in that the device comprises at least one mist generating chamber or an aerosol generating chamber.

16. Apparatus according to one of the claims 12 through 14 characterized in that the device comprises one or several nozzles or spray heads by way of which a rain can be generated.

17. Apparatus according to claim 11 characterized in that the apparatus additionally comprises a rotameter for measuring the volume streams of gas fed in, temperature measurement devices for checking a homogeneous temperature distribution, air filter for sterilization of feed in air and of exhaust air, pressure gauges, pressure measurement apparatus, mist deposit filter, and condensate catch container.

18. Apparatus according to claim 11 characterized in that the vessel for collecting the cell products is connected to a product reservoir, wherein the product reservoir is disposed outside of the supply container.

19. Use of the apparatus according to claims 11 through 18 for cultivating of cells at high densities and for obtaining of cell products, cell components, viruses or active agents.

20. Use according to claim 19 for obtaining medicine.

21. Use according to claim 29 for production of diagnostics.

22. A method for cultivation of cells at high densities and for obtaining of products from these cells comprising the following steps:
furnishing a supply container;
furnishing a cultivation chamber including several chambers and semipermeably separated from the supply container;
entering of cells of high density into the cultivation chamber;
feeding the cells of high density disposed in the cultivation chamber from the supply container with a variably adjustable gas/cell cultivation medium mixture;
generating products in the cultivation chamber with the cells of high density;
dissolving the products in the variably adjustable gas/cell cultivation medium mixture;
separating the products from the cells of high density at semipermeable walls; and
obtaining the products and discharging the products at the semipermeable walls of the cultivation chamber.

23. An apparatus for cultivation of cells at high densities and for obtaining of products from these cells, comprising
a device for generating a variably adjustable gas/ cell culture media mixture;
a supply container containing a variably adjustable gas/cell cultivation medium mixture;
a cultivation chamber is furnished with several fixed chambers, wherein the several fixed chambers are semipermeably separated from the supply container;
cells at high density disposed in the cultivation chamber for generating cell products;
vessels disposed below the cultivation chamber for collecting the cell products.
